(19) United States
(12) Patent Application Publication
(10) Pub. No.: US 2026/0026763 A1
(43) Pub. Date: Jan. 29, 2026

(54) ORGAN DEFORMATION ESTIMATION DEVICE, TREATMENT DEVICE, TREATMENT SUPPORT DEVICE, ORGAN DEFORMATION ESTIMATION METHOD, AND PROGRAM

(71) Applicants: University of Tsukuba, Tsukuba-shi, Ibaraki (JP); TOHOKU UNIVERSITY, Sendai-shi, Miyagi (JP)

(72) Inventors: Yoshihiro KURODA, Tsukuba-shi (JP); Yuki HARA, Tsukuba-shi (JP); Noriyuki KADOYA, Sendai-shi (JP); Rei UMEZAWA, Sendai-shi (JP); Keiichi JINGU, Sendai-shi (JP)

(21) Appl. No.: 18/998,016

(22) PCT Filed: Jul. 11, 2023
(86) PCT No.: PCT/JP2023/025588

§ 371 Date: Jan. 23, 2025

(30) Foreign Application Priority Data
Jul. 27, 2022 (JP) 2022-119514

(51) Int. Cl. A61B 6/00; A61B 6/03; G06T 7/00; G06T 7/50

(52) U.S. Cl. CPC A61B 6/486; A61B 6/03; G06T 7/0016; G06T 7/50

(57) ABSTRACT

An organ deformation estimation device includes a 3D model acquisition unit configured to acquire a 3D model generated based on a 3D image of an organ captured in advance at a time before a treatment is performed and showing a shape of the organ, a captured image acquisition unit configured to acquire a captured image in which an interior of the organ is captured in a course of the treatment, a partial image extraction unit configured to extract a partial image from the 3D image, a registration unit configured to perform registration between the captured image and the partial image, a target position calculation unit configured to calculate a target position based on a result of the registration, and a displacement estimation unit configured to estimate deformation of the organ in the course of the treatment by deforming the 3D model based on a 3D simulation so as to displace a position of the part that corresponds to the interior among parts constituting the 3D model to the target position.

### CROSS-REFERENCE TO RELATED APPLICATIONS

This application is the U.S. National Stage entry of International Application No. PCT/JP2023/025588, filed on Jul. 11, 2023, which, in turn, claims priority to Japanese Patent Application No. 2022-119514, filed on Jul. 27, 2022, both of which are hereby incorporated herein by reference in their entireties for all purposes.

### TECHNICAL FIELD

The present invention relates to an organ deformation estimation device, a treatment device, a treatment support device, an organ deformation estimation method, and a program.

### BACKGROUND ART

Radiotherapy is now widely used in a cancer treatment because radiotherapy allows a treatment without the need for scalpels in the body. On the other hand, outcomes in radiotherapy depend on the intensity of the radiation. Some types of organs cannot be exposed to high doses of radiation. For example, the pancreas is an organ that is located mostly behind the stomach and ranges widely from the duodenum to the spleen. In addition, the pancreas and duodenum have a particularly close relationship because the opening of the pancreatic duct is located in the duodenum. Therefore, the pancreas cannot be irradiated with high doses of radiation due to the risk of accidental irradiation to other organs, making radiotherapy for pancreatic cancer more difficult than for cancer of other organs.

Recently, a device that performs radiotherapy while taking real-time magnetic resonance (MR) images (MR-Linac) has been put to practical use, and the treatment of a pancreas with a high radiation dose is expected. However, only a few cross sections can be captured in real time, and it is difficult to modify pre-planned labels to match the movement of the organs while taking into account contact with surrounding organs and spontaneous deformation. Therefore, it is necessary to estimate the displacement of the entire model from some images.

Research on MR image-guided radiotherapy is active. For example, there are known technologies for estimating organ displacement by machine learning (Patent Document 1 or Non-patent Document 1). The technology, which uses machine learning to estimate organ displacement, represents a wide variety of movements by simultaneously learning data from a large number of patients.

### CITATION LIST

### Patent Document

Patent Document 1: PCT International Publication No. WO2020/054503

### Non Patent Document

Non-Patent Document 1: “Medical Image Computing and Computer Assisted Intervention-MICCAI 2021, Lecture Notes in Computer Science”, 2021 Sep. 21, vol. 12904, pp. 238 to 248

### SUMMARY OF INVENTION

### Technical Problem

However, the deformation of the pancreas, which is affected by a plurality of surrounding organs, is non-cyclical and varies from patient to patient. This makes it difficult to extract features by using machine learning, and it is thought that a huge amount of data set will be required for learning. In the course of a treatment, it is necessary to be able to estimate organ displacement from a small number of captured images.

The present invention was made in view of the above, and provides an organ deformation estimation device, a treatment device, a treatment support device, an organ deformation estimation method, and a program that can estimate organ displacement from a small number of captured images in the course of a treatment.

### Solution to problem

The present invention has been made in order to solve the above-described problems, and according to one aspect of the present invention, there is provided an organ deformation estimation device including a 3D model acquisition unit configured to acquire a 3D model generated based on a 3D image of an organ captured in advance at a time before a treatment is performed and showing a shape of the organ at the time before the treatment is performed, a captured image acquisition unit configured to acquire a captured image which is a 2D image in which an interior of the organ is captured in a course of the treatment, a partial image extraction unit configured to extract, from the 3D image, a partial image which is a 2D image of a part that corresponds to a position of the interior captured in the captured image among parts included in the 3D image, a registration unit configured to perform registration between pixels constituting the captured image and pixels constituting the partial image such that pixels indicating the same part of the interior correspond to each other, a target position calculation unit configured to calculate a target position of displacement of a part that corresponds to the interior among parts constituting the 3D model based on a result of the registration, and a displacement estimation unit configured to estimate deformation of the organ in the course of the treatment by deforming the 3D model based on a 3D simulation so as to displace a position of the part that corresponds to the interior among parts constituting the 3D model to the target position.

In addition, in the organ deformation estimation device according to one aspect of the present invention, the organ consists of a target organ of the treatment and other organs adjacent to the target organ.

In addition, the organ deformation estimation device according to one aspect of the present invention further includes a 3D image acquisition unit that acquires the 3D image.

In addition, in the organ deformation estimation device according to one aspect of the present invention, the displacement estimation unit performs the 3D simulation based on a mesh-free method.

In addition, the organ deformation estimation device according to one aspect of the present invention further includes a tomogram selection unit that selects a captured tomogram to be captured as the captured image of the interior of the organ.

In addition, in the organ deformation estimation device according to one aspect of the present invention, the tomogram selection unit selects the captured tomogram, from among candidate captured tomograms for one or more tomogram directions and one or more positions in the tomogram directions, based on an error of an estimation result of deformation of the organ estimated in a case where a 2D image acquired for the candidate tomograms from correct data, which is a 3D image obtained by randomly deforming the 3D image, is used instead of the captured image with respect to the correct data.

In addition, according to one aspect of the present invention, there is provided a treatment device including the organ deformation estimation device.

In addition, according to one aspect of the present invention, there is provided a treatment support device including the organ deformation estimation device.

In addition, according to another aspect of the present invention, there is provided an organ deformation estimation method including a 3D model acquisition step of acquiring a 3D model generated based on a 3D image of an organ captured in advance at a time before a treatment is performed and showing a shape of the organ at the time before the treatment is performed, a captured image acquisition step of acquiring a captured image which is a 2D image in which an interior of the organ is captured in a course of the treatment, a partial image extraction step of extracting, from the 3D image, a partial image which is a 2D image of a part that corresponds to a position of the interior captured in the captured image among parts included in the 3D image, a registration step of performing registration between pixels constituting the captured image and pixels constituting the partial image such that pixels indicating the same part of the interior correspond to each other, a target position calculation step of calculating a target position of displacement of a part that corresponds to the interior among parts constituting the 3D model based on a result of the registration, and a displacement estimation step of estimating deformation of the organ in the course of the treatment by deforming the 3D model based on a 3D simulation so as to displace a position of the part that corresponds to the interior among parts constituting the 3D model to the target position.

In addition, according to still another aspect of the present invention, there is provided a program for causing a computer to execute a 3D model acquisition step of acquiring a 3D model generated based on a 3D image of an organ captured in advance at a time before a treatment is performed and showing a shape of the organ at the time before the treatment is performed, a captured image acquisition step of acquiring a captured image which is a 2D image in which an interior of the organ is captured in a course of the treatment, a partial image extraction step of extracting, from the 3D image, a partial image which is a 2D image of a part that corresponds to a position of the interior captured in the captured image among parts included in the 3D image, a registration step of performing registration between pixels constituting the captured image and pixels constituting the partial image such that pixels indicating the same part of the interior correspond to each other, a target position calculation step of calculating a target position of displacement of a part that corresponds to the interior among parts constituting the 3D model based on a result of the registration, and a displacement estimation step of estimating deformation of the organ in the course of the treatment by deforming the 3D model based on a 3D simulation so as to displace a position of the part that corresponds to the interior among parts constituting the 3D model to the target position.

### Advantageous Effects of Invention

According to the present invention, it is possible to estimate organ displacement from a small number of captured images in the course of treatment.

### BRIEF DESCRIPTION OF DRAWINGS

FIG. 1 A diagram showing an overview of organ deformation estimation processing according to a first embodiment of the present invention.

FIG. 2 A diagram showing an overview of a material point method according to the first embodiment of the present invention.

FIG. 3 A diagram showing an example of a functional configuration of an organ deformation estimation system according to the first embodiment of the present invention.

FIG. 4 A diagram showing an example of a flow of the organ deformation estimation processing according to the first embodiment of the present invention.

FIG. 5 A diagram showing an example of an overall algorithm of a 3D simulation according to the first embodiment of the present invention.

FIG. 6 A diagram showing parameters according to an example of the first embodiment of the present invention.

FIG. 7 A diagram showing a 3D model before being driven according to the example of the first embodiment of the present invention.

FIG. 8 A diagram showing the 3D model after being driven according to the example of the first embodiment of the present invention.

FIG. 9 A diagram showing one example of the result of applying the organ deformation estimation processing according to the first embodiment of the present invention, which is high in accuracy.

FIG. 10 A diagram showing one example of the result of applying the organ deformation estimation processing according to the first embodiment of the present invention, which is low in accuracy.

FIG. 11 A box plot showing a distribution of errors from the result of the organ deformation estimation processing according to the example of the first embodiment of the present invention.

FIG. 12 A diagram showing an example of a functional configuration of an organ deformation estimation system according to a second embodiment of the present invention.

FIG. 13 A diagram showing an example of a flow of tomogram selection processing in a case where tomograms are finite and can be fully searched according to the second embodiment of the present invention.

FIG. 14 A diagram showing an example of the tomogram selection processing in a case where the tomograms are finite and cannot be fully searched or in a case where the tomograms are not finite according to the second embodiment of the present invention.

FIG. 15 A diagram showing errors and tomogram numbers for each of five cases of correct data according to the example of the second embodiment of the present invention.

FIG. 16 A diagram showing a graph of a transition of an error in a case where the tomogram number is changed according to the example of the second embodiment of the present invention.

FIG. 17 A diagram showing a graph of a transition of an error in a case where the tomogram number is changed according to the example of the second embodiment of the present invention.

FIG. 18 A diagram showing a graph of a transition of an error in a case where the tomogram number is changed according to the example of the second embodiment of the present invention.

FIG. 19 A diagram showing a graph of a transition of an error in a case where the tomogram number is changed according to the example of the second embodiment of the present invention.

FIG. 20 A diagram showing a graph of a transition of an error in a case where the tomogram number is changed according to the example of the second embodiment of the present invention.

FIG. 21 A diagram showing a graph showing the relationship between the transition of error and the number of particles in a case where the tomogram number is changed according to the example of the second embodiment of the present invention.

FIG. 22 A diagram showing a graph showing the relationship between the transition of error and the number of particles in a case where the tomogram number is changed according to the example of the second embodiment of the present invention.

FIG. 23 A diagram showing a graph showing the relationship between the transition of error and the number of particles in a case where the tomogram number is changed according to the example of the second embodiment of the present invention.

FIG. 24 A diagram showing a graph showing the relationship between the transition of error and the number of particles in a case where the tomogram number is changed according to the example of the second embodiment of the present invention.

FIG. 25 A diagram showing a graph showing the relationship between the transition of error and the number of particles in a case where the tomogram number is changed according to the example of the second embodiment of the present invention.

### DESCRIPTION OF EMBODIMENTS

### First Embodiment

Hereinafter, embodiments of the present invention will be described in detail with reference to drawings. In the following description, estimating the displacement of each part constituting an organ as a 3D shape is referred to as estimating the deformation of the shape of an organ, or estimating the deformation of an organ, and the like. In addition, the cross-section of an organ is also referred to as the tomogram of an organ. In the present embodiment, the processing of estimating the deformation of the shape of an organ is referred to as organ deformation estimation processing. In addition, in each embodiment, an example of a case where organ deformation estimation processing is applied to radiotherapy is described, but the organ deformation estimation processing may also be applied to treatments other than radiotherapy.

### [Overview of Organ Deformation Estimation Processing]

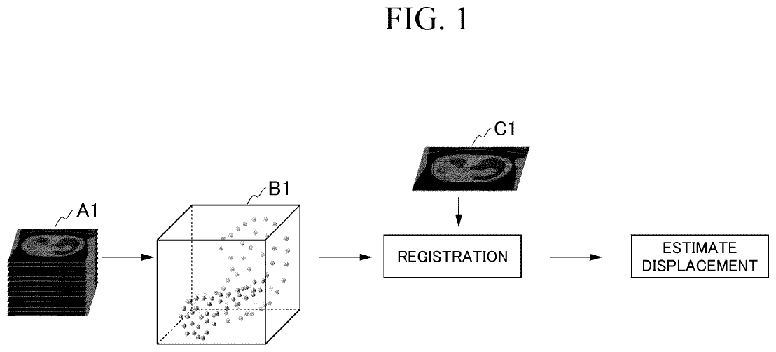

FIG. 1 is a diagram showing an overview of organ deformation estimation processing according to the present embodiment. In the organ deformation estimation processing, a 3D image A1 of an organ is captured in advance before the radiotherapy is performed. A 3D model B1 of the organ is generated from the 3D image A1. In the organ deformation estimation processing, the 3D model B1 is driven by a 3D simulation. A captured image C1 is used when the 3D model B1 is driven by a 3D simulation. The captured image C1 is a 2D image in which the tomogram of the organ is captured during radiotherapy. Registration is performed between the 2D image of the 3D images A1 that corresponds to a tomogram captured in the captured image C1 and the captured image C1. The registration calculates the displacement of the position of each part of the tomogram from the pre-treatment position thereof due to deformation of the organ during the treatment. In the organ deformation estimation processing, the part of the 3D model B1 corresponding to the tomogram captured in the captured image C1 is displaced by the 3D simulation by the displacement calculated by the registration. The parts of the 3D model B1 other than the part corresponding to the tomogram are displaced following the displacement of the part corresponding to the tomogram. As a result, the overall displacement of the 3D shape of the organ is estimated from the captured image C1, in which the tomogram of the organ is captured.

In the present embodiment, the pancreas is treated as an example of a target organ whose deformation is to be estimated. The pancreas is adjacent to surrounding organs, and it is generally difficult to estimate the deformation of the organ while taking into account contact with surrounding organs as well as spontaneous movement. In the organ deformation estimation processing, the deformation of the shape of the pancreas is estimated with high accuracy by taking into account the fact that the pancreas is adjacent to the surrounding organs. In the organ deformation estimation processing, the pancreas and surrounding organs are each modeled as a 3D model and simulated in 3D. In the organ deformation estimation processing, the position information of the pancreas and surrounding organs obtained from the tomographic image is used to estimate the deformation of the shape of the organ by performing a 3D simulation that takes into account the contact between the pancreas and the surrounding organs.

In the organ deformation estimation processing, the material point method (MPM) is used to perform a 3D simulation that takes into account contact between the pancreas and surrounding organs. The MPM is one of the mesh-free methods, in which each part of an object is discretized by replacing the part with particles, while the calculation is performed by using a lattice prepared separately from the particles. The MPM has the advantage that contact between different objects can be handled in the natural flow of the algorithm, since physical quantities are calculated after all particle information is transferred to the lattice points.

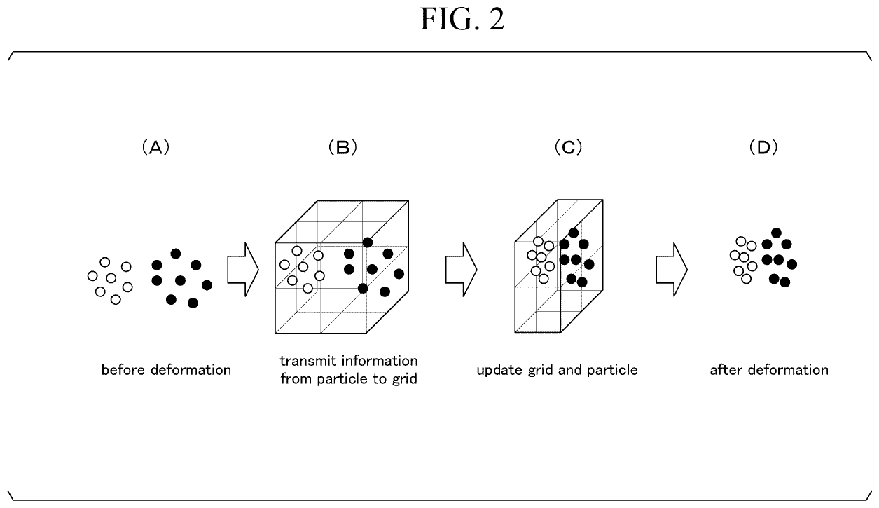

FIG. 2 is a diagram showing an overview of the MPM according to the present embodiment. The MPM uses particles to track the position, mass, velocity, and deformation gradient of an object or the like. In other words, particles carry the physical information of an object. On the other hand, the MPM uses lattice points to update particle information based on conservation laws and constitutive laws. The constitutive laws depend on the target object. On the other hand, conservation laws are common to all objects, and the laws of conservation of mass, momentum, and angular momentum are used. The laws of conservation of mass and the laws of conservation of momentum are expressed by the following Equations (1) and (2), respectively.

$$∇ \cdot v = 0 \qquad ( 1 )$$

$$ρ \frac{Dv}{Dt} = ∇ \cdot σ + ρ g \qquad ( 2 )$$

Here, ρ is density, t is time, v is velocity, σ is stress, and g is gravitational acceleration. The laws of conservation of angular momentum are guaranteed because the stress tensor is symmetric. These conservation laws transfer the physical information carried by the particles to the lattice points and are solved on the lattice. Therefore, the MPM is a calculation method that combines both Eulerian and Lagrangian perspectives. In addition, the MPM also has the advantage of particle methods because the object is discretized into particles.

FIG. 2(A) shows the positions of the particles before deformation. FIG. 2(B) shows the physical information carried by the particles is transferred to the lattice points. FIG. 2(C) shows a state in which the positions of the lattice points are updated based on the conservation laws and the constitutive laws, and the positions of the particles are updated based on the physical information transferred from the lattice points to the particles. FIG. 2(D) shows the positions of the particles after deformation.

### [Functional Configuration of Organ Deformation Estimation System 1]

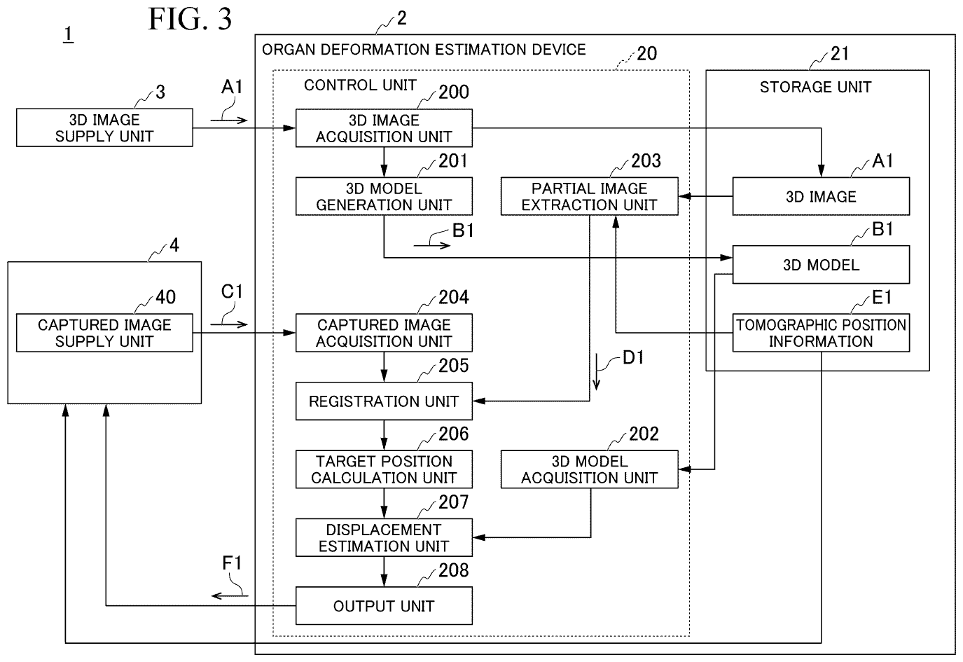

FIG. 3 is a diagram showing an example of a functional configuration of an organ deformation estimation system 1 according to the present embodiment. The organ deformation estimation system 1 includes an organ deformation estimation device 2, a 3D image supply unit 3, and a captured image supply unit 40. The captured image supply unit 40 is provided in a radiotherapy device 4.

The organ deformation estimation device 2 performs organ deformation estimation processing. The organ deformation estimation device 2 is a computer, such as a personal computer (PC), workstation, or server, as an example. The organ deformation estimation device 2 is, as an example, a computer that is separate from the radiotherapy device 4, but may also be integrated with the radiotherapy device 4 by being built into the console of the radiotherapy device 4 or the like.

The 3D image supply unit 3 supplies the 3D image A1 to the organ deformation estimation device 2. The 3D image supply unit 3 is a medical imaging device. The 3D image supply unit 3 is, for example, a nuclear magnetic resonance imaging (MRI) device.

The radiotherapy device 4 is a device that performs radiotherapy while taking real-time magnetic resonance (MR) images (MR-Linac). Therefore, the radiotherapy device 4 includes the function as a medical imaging device.

In the present embodiment, the 3D image supply unit 3 is separate from the radiotherapy device 4, but the 3D image supply unit 3 may be included in the radiotherapy device 4.

The 3D image supply unit 3 may be a computed tomography (CT) device. In addition, the radiotherapy device 4 may also be a device that performs radiotherapy while taking CT images in real time. In addition, the radiotherapy device 4 may also be a device (Linac) that performs radiotherapy while imaging the organ to be treated with X-rays in real time.

The organ deformation estimation device 2 includes a control unit 20 and a storage unit 21.

The control unit 20 includes, for example, a central processing unit (CPU), graphics processing unit (GPU), or field-programmable gate array (FPGA), random access memory (RAM), and the like, and performs various calculations and exchange of information. Each functional part provided by the control unit 20 is realized by a central processing unit (CPU) reading a program from read only memory (ROM) and executing the processing. The ROM is included in the storage unit 21.

The control unit 20 includes a 3D image acquisition unit 200, a 3D model generation unit 201, a 3D model acquisition unit 202, a partial image extraction unit 203, a captured image acquisition unit 204, a registration unit 205, a target position calculation unit 206, a displacement estimation unit 207, and an output unit 208.

The 3D image acquisition unit 200 acquires the 3D image A1 from the 3D image supply unit 3. The 3D image A1 is a 3D image of an organ captured in advance at a time before the organ is irradiated with radiation in radiotherapy. In the present embodiment, the organs consist of a pancreas, which is the organ to be treated by 15 radiotherapy, and other organs adjacent to the pancreas (surrounding organs). The 3D image acquisition unit 200 stores the acquired 3D image A1 in the storage unit 21. Other organs adjacent to the organ to be treated by radiotherapy (surrounding organs) may or may not be in contact with the organ to be treated by radiotherapy.

The 3D model generation unit 201 generates the 3D model B1 based on the 3D image A1. The 3D model B1 is a 3D model showing the shape of the organ at a time before radiotherapy is performed. The 3D model generation unit 201 stores the generated 3D model B1 in the storage unit 21.

The 3D model acquisition unit 202 acquires the 3D model B1 generated by the 3D model generation unit 201. The 3D model acquisition unit 202 supplies the acquired 3D model B1 to the displacement estimation unit 207.

The 3D model B1 may be generated by a computer that is separate from the organ deformation estimation device 2. In that case, the 3D model generation unit 201 may be omitted from the configuration of the organ deformation estimation device 2, and the 3D model acquisition unit 202 acquires the 3D model B1 from the computer.

The partial image extraction unit 203 extracts a partial image D1 from the 3D image A1.

The partial image D1 is a 2D image of the part that corresponds to the position of the interior of an organ captured in the captured image C1 among parts included in the 3D image A1. In the present embodiment, the interior of an organ is the tomogram of the organ.

In the present embodiment, the position of the tomogram of the organ is predetermined. Information indicating the position of the tomogram is stored in the storage unit 21 in advance as tomographic position information E1. The tomogram of the organ is, for example, a cross-section passing through the center of gravity of the organ (pancreas) to be treated by radiotherapy. The cross-section in the direction through the center of gravity is arbitrary, but can be, for example, a cross-section in any of the three axes in a case where 3D Cartesian coordinates are set, a cross-section of a patient's body axis, or a coronal cross-section or a sagittal cross-section. The tomogram of the organ may be determined so that the area of the tomogram of the organ (pancreas) to be treated by radiotherapy is the largest. In addition, the direction passing through the center of gravity may be the direction that includes the position at which the organ (pancreas) to be treated by radiotherapy is in contact with the surrounding organs. In addition, the direction passing through the center of gravity may be the direction in which the distance between the parts (particles) closest to each other for the part constituting the organ (pancreas) to be treated by radiotherapy and the parts constituting the surrounding organs is the shortest.

In addition, the position of the organ tomogram may also be determined so that the tomogram contains more types (number) of organs (labels of organs). Further, the position of the organ tomogram may also be determined so that the tomogram contains each organ as evenly as possible with respect to area.

The captured image acquisition unit 204 acquires the captured image C1. The captured image C1 is a 2D image in which the interior of an organ (tomogram) is captured in the course of radiotherapy. In the present embodiment, the captured image C1 is a tomographic image in which the tomogram of an organ is captured.

The registration unit 205 performs registration between the pixels constituting the captured image C1 and the pixels constituting the partial image D1 such that pixels indicating the same part of the interior of an organ (tomogram) correspond to each other.

The target position calculation unit 206 calculates the target position of displacement for the part that corresponds to the interior of an organ (tomogram) among parts constituting the 3D model B1 based on the results of the registration by the registration unit 205.

The displacement estimation unit 207 estimates the deformation of the organ in the course of the radiotherapy based on the 3D simulation. Here, the displacement estimation unit 207 estimates the deformation of the organ in the course of the radiotherapy by deforming the 3D model B1 based on the 3D simulation so as to displace the position of the part that corresponds to the interior (tomogram) among parts constituting the 3D model B1 to the target position calculated by the target position calculation unit 206. The displacement estimation unit 207 performs a 3D simulation based on a mesh-free method. The mesh-free method is, for example, MPM.

The output unit 208 outputs the result of the organ deformation estimated by the displacement estimation unit 207 to the radiotherapy device 4 as an estimation result F1. The estimation result F1 includes, for example, one or more of the following: a tomographic image after deformation, a 3D model after deformation, and a 3D label after deformation. The 3D label indicates the target position in the organ irradiated with radiation. The 3D model includes, for example, pixel value information of the 3D image A1 and 3D label information. Therefore, in a case of outputting a 3D model, it is equivalent to outputting a tomographic image and 3D label information after deformation as well. The radiotherapy device 4 changes the pre-planned 3D labels in real time in the course of the radiotherapy based on the estimation result F1.

The storage unit 21 stores various information. The information stored in the storage unit 21 includes the 3D image A1, the 3D model B1, and the tomographic position information E1. The storage unit 21 is configured by using a storage device such as a magnetic hard disk device or a semiconductor storage device.

The organ deformation estimation device 2 and the 3D image supply unit 3 may be connected by a cable for communication, or may communicate through a wireless network such as a local area network (LAN). The 3D image A1 captured by the 3D image supply unit 3 may be stored in an external storage device and then supplied to the organ deformation estimation device 2 via the external storage device. In that case, the 3D image A1 may be transferred from the external storage device to the organ deformation estimation device 2 by a user.

The organ deformation estimation device 2 and the radiotherapy device 4 may be connected by a cable for communication or may communicate through a wireless network such as a LAN.

### [Organ Deformation Estimation Processing]

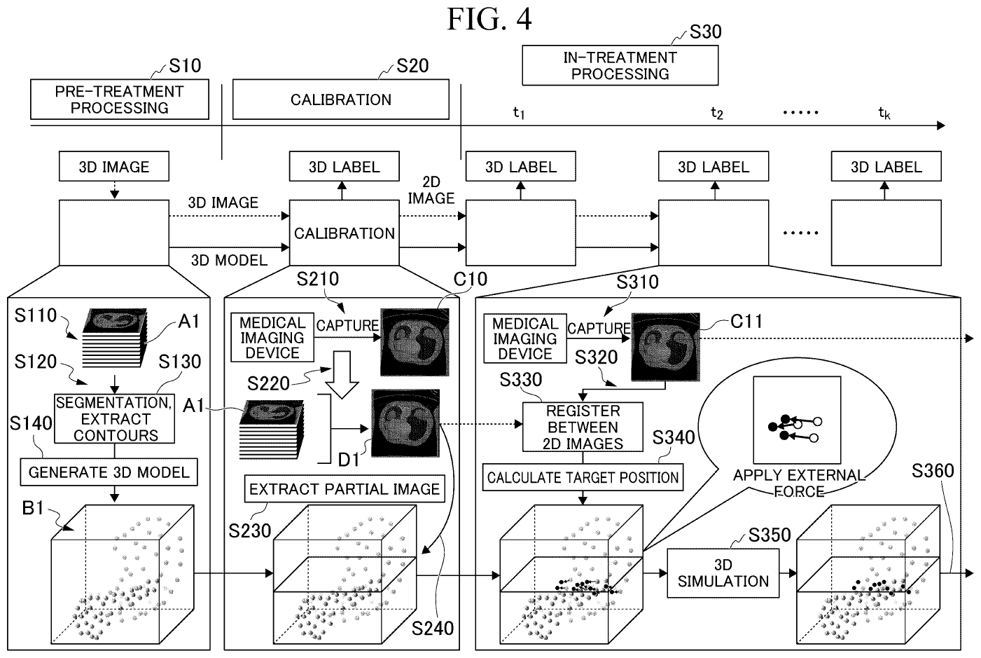

FIG. 4 is a diagram showing an example of a flow of organ deformation estimation processing according to the present embodiment. The organ deformation estimation processing includes pre-treatment, calibration, and in-treatment processing.

Step S10: The organ deformation estimation system 1 executes the pre-treatment processing. The pre-treatment is executed at a time before the radiotherapy is performed. The pre-treatment is processing of creating the 3D model B1 from the 3D image A1. The time before the radiotherapy is performed is, for example, one day before the radiotherapy is performed. It is required that the condition of the organ remains as unchanged as possible between the time before radiotherapy and the course of the radiotherapy. Therefore, it is preferable that pre-treatment be performed as soon before radiotherapy as possible. In addition, the pre-treatment processing may be performed, for example, after the start of radiotherapy, so long as the pre-treatment processing is performed before radiation is irradiated onto the organ in radiotherapy. In that case, the 3D image A1 is captured in advance at a time after radiotherapy is started, but before calibration is performed.

Each of the processing from step S110 to step S140 is performed as pre-treatment processing.

Step S110: The 3D image supply unit 3 captures the 3D image A1.

The 3D image supply unit 3 captures the 3D image A1 of an organ by, for example, MRI.

Step S120: The 3D image acquisition unit 200 acquires the 3D image A1 from the 3D image supply unit 3.

Step S130: The 3D model generation unit 201 extracts the contours of the organs from the 3D image A1 based on one or more kinds of processing of segmentation and contouring. In the present embodiment, the contour of each of the pancreas and the stomach as a surrounding organ is extracted. The contours include the external contours of the organ and the internal contours of the organ.

Step S140: The 3D model generation unit 201 generates the 3D model B1 based on the extracted contour of the organ. In the present embodiment, the 3D model B1 is a 3D model of the pancreas and stomach. The 3D model B1 is, as an example, a 3D model in which each part of an organ is replaced by a particle and the shape of the organ is discretized. In a case where a plurality of organs are included in the 3D model B1, it is possible to identify in the 3D model B1 which of the plurality of organs the particle included in the 3D model B1 is a part of, by assigning a label or the like indicating the organ to the particle. In an example of the present embodiment, it is possible to identify which organ part of the pancreas or stomach is the particle in the 3D model B1. The 3D model generation unit 201 stores the generated 3D model B1 in the storage unit 21.

Step S20: The organ deformation estimation system 1 executes calibration. The calibration is executed at the initial stage of the radiotherapy. The initial stage of the radiotherapy is, for example, immediately after the radiotherapy is started. The 3D image A1 and the captured image C1, which is captured in the course of the treatment, are captured by different devices (in the present embodiment, the 3D image supply unit 3 and the radiotherapy device 4). In addition, in the 3D image A1 and the captured image C1, which is captured in the course of the treatment, the organ may be captured in different postures. Therefore, in order to perform a 3D simulation of an organ, the captured image C1 which is captured in the course of the treatment and the 3D model B1 correspond to each other with respect to the position of each part of the organ. In the calibration, the part that corresponds to the tomogram captured in the captured image C10 among parts constituting the 3D model B1 is selected.

As the calibration, each processing of step S210 to step S240 is executed.

Step S210: The radiotherapy device 4 captures the captured image C10 at the initial stage of the radiotherapy. The radiotherapy device 4 captures the tomogram of an organ as the captured image C10 by, for example, MRI.

Step S220: The captured image acquisition unit 204 acquires the captured image C10 from the radiotherapy device 4.

Step S230: From the 3D image A1, the partial image extraction unit 203 extracts the partial image D1 which is a 2D image of the part that corresponds to the position of the tomogram captured in the captured image C10 among parts included in the 3D image A1. Here, the partial image extraction unit 203 reads the 3D image A1 and the tomographic position information E1 from the storage unit 21. The partial image extraction unit 203 selects the part that corresponds to the position of the interior of an organ captured in the captured image C10 among parts included in the 3D image A1 based on the read-out 3D image A1 and the tomographic position information E1 and the captured image C10 acquired by the captured image acquisition unit 204. The part included in the 3D image A1 is selected as the 2D image included in the 3D image A1. The processing of selecting such parts may include, for example, processing of determining a direction in the 3D image A1 that corresponds to the direction of a normal to the plane of the tomogram captured in the captured image C10.

Step S240: The partial image extraction unit 203 selects the part that corresponds to the tomogram captured in the captured image C10 among parts constituting the 3D model B1.

Step S30: The organ deformation estimation system 1 executes the in-treatment processing. In the in-treatment processing, the 3D simulation of the 3D model B1 is executed. The in-treatment processing is repeatedly executed in the course of the radiotherapy. In the present embodiment, the in-treatment processing is executed in real time each time the captured image C1 of the organ tomogram is captured by the radiotherapy device 4. The in-treatment processing may not be executed for each of all the captured images C1. The in-treatment processing may be performed once for every N (N is a natural number equal to or greater than 2) times the captured image C1 is taken, for example, to reduce the processing load of the organ deformation estimation device 2. In addition, the in-treatment processing may be executed at least once in the course of the treatment. As the in-treatment processing, each processing from step S310 to step S350 is executed.

Step S310: The radiotherapy device 4 takes a captured image C11 in the course of the radiotherapy. The radiotherapy device 4 captures the tomogram of an organ as the captured image C11 by, for example, MRI.

Step S320: The captured image acquisition unit 204 acquires the captured image C11 from the radiotherapy device 4.

Step S330: The registration unit 205 performs registration between the captured image C11 and the partial image D1. Here, the registration unit 205 performs registration between the pixels constituting the captured image C11 and the pixels constituting the partial image D1 such that the pixels indicating the same part of the tomogram of the organ correspond to each other. The partial image D1 is the 2D image selected in step S230 of the calibration.

In the registration, displacement u(x) is obtained between a d-dimensional image IF(x) (where x is an element of a d-dimensional real space) and another image IM(x) (where x is also an element of a d-dimensional real space) so that Equation (3) is satisfied.

$${I}_{M} ( x + u ( x ) ) = {I}_{F} ( x ) \qquad ( 3 )$$

In other words, the registration is attributed to the problem of finding a deformation function T that satisfies Equation (4).

$${I}_{M} ( T ( x ) ) = {I}_{F} ( x ) \qquad ( 4 )$$

In practice, however, two images rarely coincide perfectly with each other, and thus the optimal deformation function T (T with a hat in Equation (5)) is obtained, as expressed in Equations (5) and (6).

$$\hat{T} = {argmin}_{T} C ( T ; {I}_{F} , {I}_{M} ) \qquad ( 5 )$$

$$C ( T ; {I}_{F} , {I}_{M} ) = - S ( T ; {I}_{F} , {I}_{M} ) + γ P ( T ) \qquad ( 6 )$$

In other words, the registration is formulated as a minimization problem with respect to a deformation function T (T with a hat in Equation (5)). In Equations (5) and (6), C is a similarity function, P is a penalty function, and γ is a weight for the penalty.

Depending on what function is assumed for the deformation function T (T with a hat in Equation (5)), there are different degrees of freedom of deformation: rigid deformation or non-rigid deformation that allows only translation and rotation. For non-rigid deformation, there are numerous models that assume an affine transformation or a B-spline transformation. In the present embodiment, as an example, the deformation function T (T with a hat in Equation (5)) is assumed to be an affine transformation, but may be a rigid transformation, a B-spline transformation, or any other transformation.

A predetermined 3D medical image registration library may be used for the registration. In the present embodiment, elastix was used as an example as a 3D medical image registration library. elastix is a toolbox that supports a plurality of medical images and can perform registration between 2D images by using various types of deformation and evaluation index, both rigid and non-rigid. Mutual Information (MI) was used as the evaluation index. By using elastix, an affine transformation matrix A and a translation vector t shown in Equation (7) can be obtained.

$${f}_{affine} ( x ) = A ( x - c ) + t + c \qquad ( 7 )$$

The affine transformation matrix A and the translation vector t are used as inputs to the 3D simulation. Here, x is a position vector of a pixel, and c is a position vector of the image center.

Step S340: The target position calculation unit 206 calculates the target position of displacement of the part that corresponds to the tomogram of the organ among parts constituting the 3D model B1 based on the result of the registration by the registration unit 205. The target position calculation unit 206 calculates the target positions of particles present in the 3D model B1 in the part that corresponds to the tomogram captured in the captured image C11. The target position calculation unit 206 uses the affine transformation matrix A and the translation vector t described above to calculate the target positions as an example.

Step S350: The displacement estimation unit 207 estimates the deformation of the pancreas in the course of the radiotherapy based on the 3D simulation by MPM. In the 3D simulation by MPM, the contact between the organs can be handled without changing the algorithm of the MPM from a case where there is no contact between the organs.

In the present embodiment, the pancreas and stomach were modeled as linear elastic bodies, and one particle was assigned per pixel. N particles may be assigned to M pixels (M, and N are natural numbers). For each particle, the 3D model B1 is driven by applying the force expressed in Equation (8) until the difference between the current position and the target position falls below a threshold value.

$$f = {K}_{p} {x}_{diff} + {K}_{i} \int {x}_{diff}^{d} t + {K}_{d} \frac{{}_{d} {x}_{diff}}{{d}_{} t} \qquad ( 8 )$$

Here, xdiff is given by Equation (9).

$${x}_{diff} = {x}_{t a r} - {x}_{c u r} \qquad ( 9 )$$

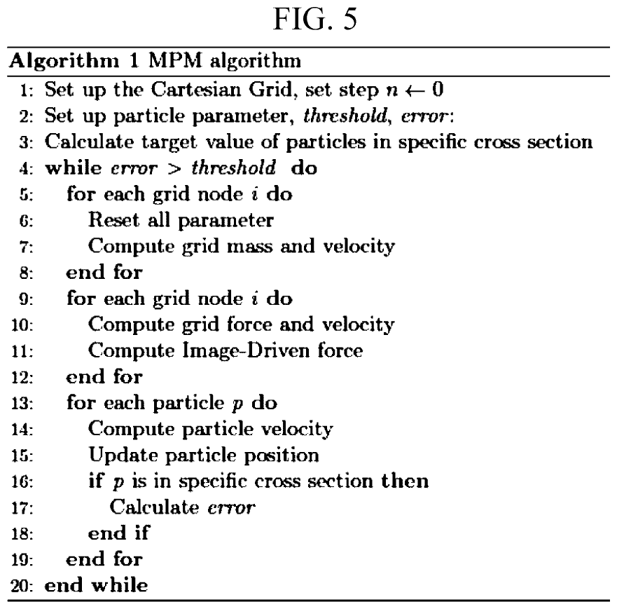

Here, xtar is the target position, xcur is the position vector of the current position, Kp is the coefficient for the proportional term, Ki is the coefficient for the integral term, and Kd is the coefficient for the differential term. The overall algorithm for the 3D simulation is shown in FIG. 5.

The 3D simulation by the displacement estimation unit 207 took into account the contact between the organ (pancreas) and the surrounding organs (stomach) to be treated by radiotherapy. Therefore, in the organ deformation estimation processing, it is possible to express deformation that is not periodic due to the positional relationship with the surrounding organs and the movement of the surrounding organs as deformation of the organs to be treated by radiotherapy.

Step S360: The output unit 208 outputs the estimation result F1 to the radiotherapy device 4. The 3D label included in the estimation result F1 indicates a pixel in the 3D image A1 in which the organ was captured that correspond to the area to be irradiated with radiation. The 3D label may be designated by using particles constituting the 3D model B1. In addition, the 3D label may include not only the label of the organ to be treated by radiotherapy, but also the labels of the surrounding organs.

In the present embodiment, an example of a case where one captured image C11 is used in one-time 3D simulation has been described, but is not limited thereto. A plurality of captured images C11 may be used for one-time 3D simulation. In that case, the plurality of captured images C11 are, for example, a plurality of tomographic images in which tomographic images in which tomograms having the same normal direction of the tomogram and different positions in the normal direction are captured, or a plurality of tomographic images in which tomograms having different normal directions of the tomogram are captured.

In the present embodiment, an example of a case where the position of the tomogram is designated in advance by the tomographic position information E1 has been described, but is not limited thereto. The position of the tomogram may be determined based on the captured image C10 taken in step S210. In that case, the position of the tomogram that was captured in the captured image C10 is determined, and the information indicating the determined position of the tomogram is referred to as the tomographic position information E1.

In addition, the tomographic position information E1 may be calculated at a time after the pre-treatment processing and before the calibration. A case where the tomographic position information E1 is calculated will be described below in a second embodiment.

In the present embodiment, an example of a case where the captured image C1 is an MR image has been described, but is not limited thereto. The captured image C1 may be a CT image. However, since the MR image has higher contrast than the CT image, it is preferable that the MR image is used as the captured image C1.

The above-described algorithm of the MPM is an example, and various modifications may be made and used. In addition, in the present embodiment, an example of a case where a 3D simulation is performed based on the MPM has been described, but is not limited thereto. The 3D simulation may be performed based on a mesh-free method other than the MPM. In addition, a 3D simulation may be performed based on a method other than the mesh-free method. In the present embodiment, the 3D model B1 is a discretized 3D model in which each part of the organ is replaced by a particle, but the 3D model B1 is modified according to the method used for a 3D simulation.

In the present embodiment, an example has been described in which an organ consists of an organ to be treated by the radiotherapy and other organs adjacent to the target organ, and a 3D simulation is performed taking into account contact between the organs accordingly, but is not limited thereto. The organ may consist solely of the organ to be treated by radiotherapy. For example, the organ may be the pancreas only.

In the present embodiment, an example of a case where the organ to be treated by radiotherapy is a pancreas has been described, but is not limited thereto. The organ to be treated by radiotherapy may be an organ other than the pancreas, such as duodenum, spleen, and brain.

In step S230 above, an X-ray image obtained by performing internal (tomographic) imaging of the organ by using X-rays may be used in order to select the part that corresponds to the position of the interior of the organ captured in the captured image C10 from the 3D image A1. In that case, from the 3D image A1, the 2D image that most closely matches the relevant X-ray image is selected as the partial image D1.

As described above, the organ deformation estimation device 2 according to the present embodiment is an organ deformation estimation device in radiotherapy, and includes the 3D image acquisition unit 200, the 3D model acquisition unit 202, the partial image extraction unit 203, the captured image acquisition unit 204, the registration unit 205, the target position calculation unit 206, and the displacement estimation unit 207. The 3D image acquisition unit 200 acquires the 3D image A1 of an organ (in the present embodiment, pancreas) that is captured in advance at a time before the organ is irradiated with radiation in radiotherapy. The 3D model acquisition unit 202 acquires the 3D model B1, which is generated based on the 3D image A1 and shows the shape of an organ (in the present embodiment, pancreas) at a time before radiotherapy is performed. The captured image acquisition unit 204 acquires the captured image C1 which is a 2D image in which the interior (in the present embodiment, tomogram) of an organ (in the present embodiment, pancreas) is captured in the course of the radiotherapy. The partial image extraction unit 203 extracts, from the 3D image A1, the partial image D1 which is a 2D image of the part that corresponds to the position of the interior (in the present embodiment, tomogram) captured in the captured image C1 among parts included in the 3D image A1. The registration unit 205 performs registration between the pixels constituting the captured image C1 and the pixels constituting the partial image D1 such that pixels indicating the same part of the interior (in the present embodiment, tomogram) correspond to each other. The target position calculation unit 206 calculates the target position of displacement of the part that corresponds to the interior (in the present embodiment, tomogram) among parts constituting the 3D model B1 based on the results of the registration by the registration unit 205. The displacement estimation unit 207 estimates the deformation of an organ (in the present embodiment, pancreas) in the course of the radiotherapy by deforming the 3D model B1 based on a 3D simulation (in the present embodiment, MPM) so that the position of the part that corresponds to the interior (in the present embodiment, tomogram) among parts constituting the 3D model B1 is displaced to the target position calculated by the target position calculation unit 206.

With this configuration, in the organ deformation estimation device 2 according to the present embodiment, it is possible to estimate the deformation of an organ (in the present embodiment, pancreas) in the course of the radiotherapy by deforming the 3D model B1 based on a 3D simulation (in the present embodiment, MPM) so that the position of the part that corresponds to the interior (in the present embodiment, tomogram) of the organ among parts constituting the 3D model B1 is displaced to the target position calculated by using the captured image C1 in which the interior (in the present embodiment, tomogram) of the organ (in the present embodiment, pancreas) is captured in the course of the radiotherapy. Therefore, in the organ deformation estimation device 2 according to the present embodiment, it is possible to estimate the displacement of the organ from a small number of captured images in the course of the radiotherapy. The small number of images is the number of images that can be captured in the course of the radiotherapy, and is one to several images (about two to three images, or the like). Radiotherapy is an example of a treatment. The time before the organ is irradiated with radiation in radiotherapy is an example of a time before the treatment is performed. In addition, the 3D image acquisition unit 200 may be omitted from the configuration of the organ deformation estimation device 2.

In the related art, in a device known as MR-Linac, which performs radiotherapy while capturing MR images of tomograms of an organ in real time, only a few tomograms can be captured in real time during treatment. The organ deformation estimation device 2 according to the present embodiment is suitably used for estimating the displacement of an organ in the course of radiotherapy in a case where only a few tomographic images can be captured, and for changing the 3D labels for emitting the radiation in real time.

### First Example

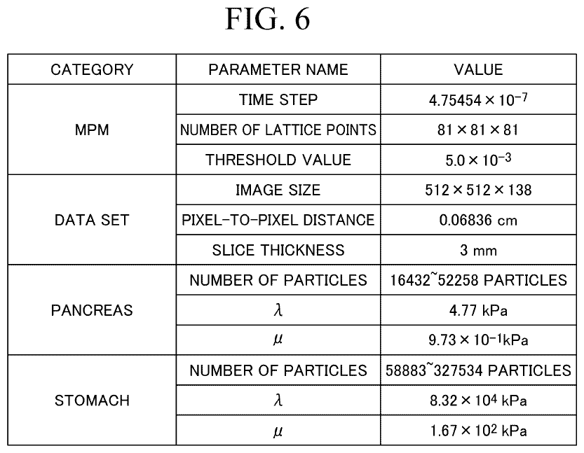

A first example which is an example according to a first embodiment will be described. In the present example, as the captured image C1 used for registering between 2D images, one tomographic image in which a tomogram at the center of a volume in which an organ was present was captured was used. The Multi-Atlas Labeling Beyond the Cranial Vault Segmentation Challenge Dataset was used for the images of an organ. MATLAB (registered trademark) was used for data processing for executing a 3D simulation. The parameters used in the present example are shown in FIG. 6.

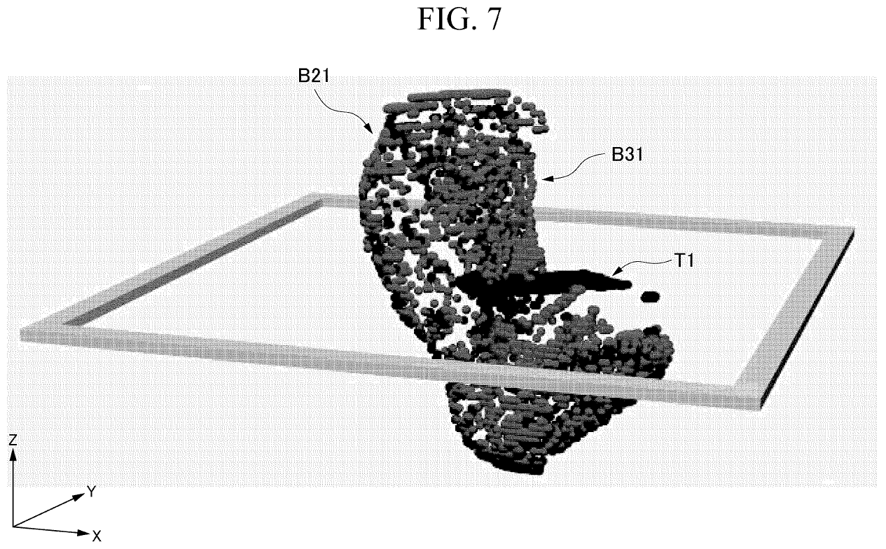

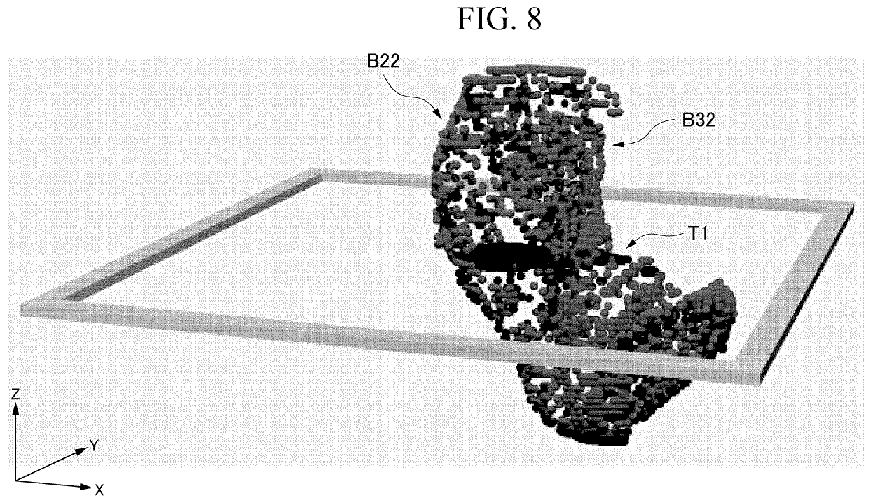

In addition, FIGS. 7 and 8 show a state in which the 3D model B1 is driven by a 3D simulation. FIGS. 7 and 8 each show a 3D Cartesian coordinate system (XYZ coordinate system). In the 3D orthogonal coordinate system, a Z-axis direction is a direction perpendicular to a tomogram, and an X-axis direction and a Y-axis direction are directions parallel to a plane of the tomogram, respectively.

FIG. 7 shows a 3D model before being driven. The 3D model B21 is a 3D model of the pancreas before being driven. The 3D model B31 is a 3D model of the stomach before being driven. FIG. 8 shows a 3D model B12 after being driven. A 3D model B22 is a 3D model of the pancreas after being driven. A 3D model B32 is a 3D model of the stomach after being driven.

A target position T1 indicates a target position for displacing a part that corresponds to the tomogram of the 3D model B11. When the part that corresponds to the tomogram is displaced to the target position T1 by the 3D simulation using the MPM, the parts other than the part that corresponds to the tomogram of the 3D model B11 are also displaced to follow the displacement.

In the present example, ground truth (GT) was created by deforming each particle constituting the 3D model B1 by applying a random force in the in-plane direction. An error & used in the present example is expressed by Expression (10).

$$ε = \frac{1}{n} {\sum }_{i = 1}^{n} ‖ {x}_{gt , i} - {x}_{est , i} ‖ \qquad ( 10 )$$

Here, n is the total number of particles in a physical model, xgt,i is the position vector in GT of the i-th particle, and xest,i is the position vector of the i-th particle whose position is estimated by the organ deformation estimation processing.

The accuracy of 3D displacement estimation was verified by using only one tomographic image by comparing the error of the organ deformation estimation processing with the results of the registration between the 3D images by elastix. In addition, a 3D simulation was performed by using the organ deformation estimation processing in a case where the stomach, a surrounding organ, was not discretized, and the presence or absence of improvement in accuracy by taking the surrounding organs into account was verified.

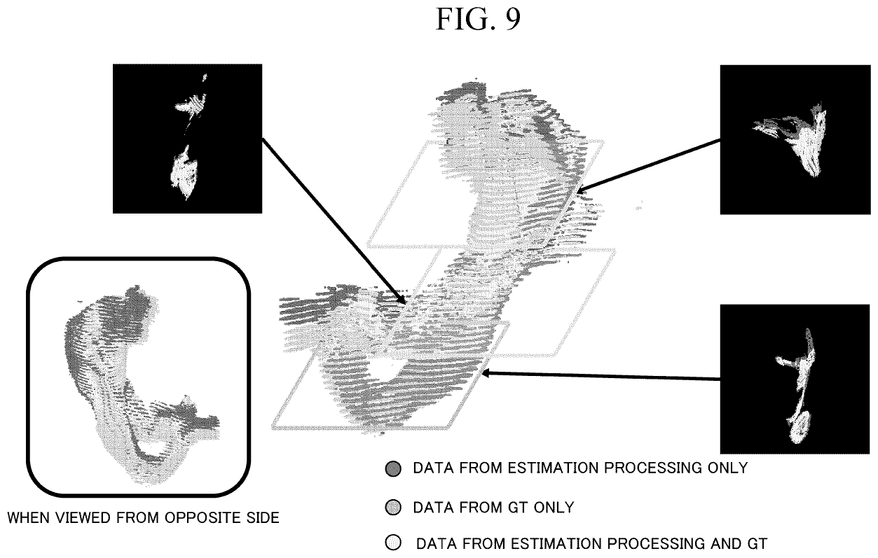

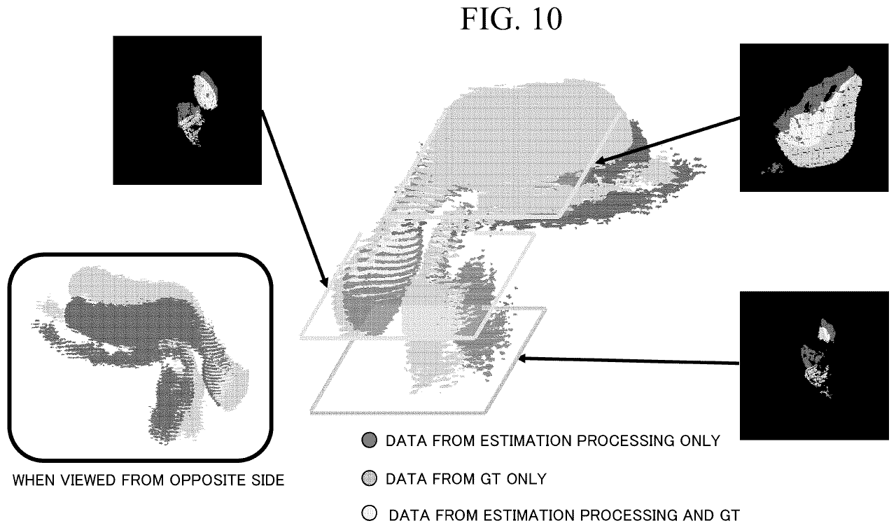

In the present example, the organ deformation estimation processing was applied to data of 20 cases. FIGS. 9 and 10 show the results of one example with high accuracy and one example with low accuracy among the data of 20 cases, respectively. In FIGS. 9 and 10, “data from the estimation processing only” is data that indicates positions estimated by the organ deformation estimation processing that are not included in the GT. The “data from GT only” is data that indicates the positions of the GT data that have not been estimated by the organ deformation estimation processing. The “data from the estimation processing and GT” is the data that indicates the positions estimated by the organ deformation estimation processing that are included in the GT.

From FIG. 9, it can be seen that the displacement can be estimated with high accuracy although some errors are found on the surface area. It is thought that the errors found on the surface area are due to the fact that only the contact with other organs is defined as a boundary condition. It is thought that the errors could be reduced by increasing the number of surrounding organs to be considered or by changing the contact algorithm to one with higher accuracy. From FIG. 10, it can be seen that an error occurs particularly at both ends of the organ. In the present example, tomographic images of the center of the region where the pancreas exists were used in order to standardize the experimental conditions. It is thought that the axial direction of the tomogram to be used and which tomogram to select in each axial direction are important.

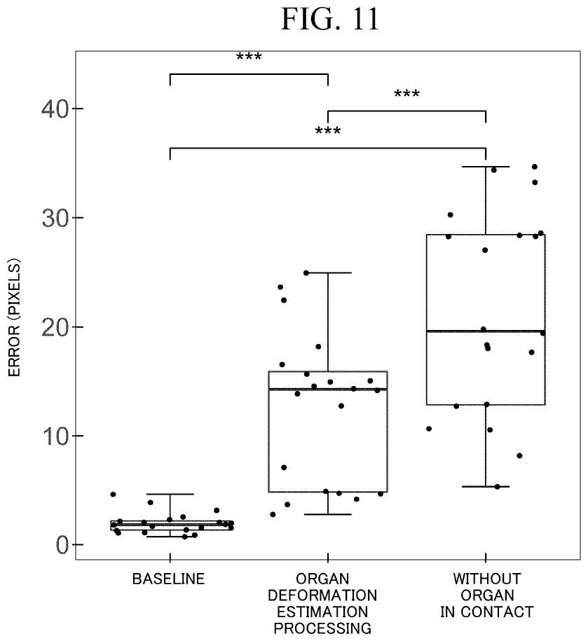

FIG. 11 shows the errors from the result of the organ deformation estimation processing for the data of 20 cases. FIG. 11 is a box plot showing the distribution of the errors in the 20 cases. The position error of the pancreas from the organ deformation estimation processing was 12.7±6.93 (pixels). In the organ deformation estimation processing, the position error of the pancreas in a case where the contact with the surrounding organs was not taken into account was 21.3±9.30 (pixels). The position error of the pancreas from the results of the registration between the 3D images was 1.97±0.981 (pixels). In terms of the average value, it was not possible to achieve an accuracy close to the results of the registration between the 3D images. However, from FIG. 11, it can be seen that the accuracy close to the results of the registration between the 3D images can be realized in the example of about 40% of the result of the organ deformation estimation processing. If an appropriate tomogram can be selected, it is possible to realize accuracy close to the results of the registration between the 3D images by the organ deformation estimation processing. A method of selecting an appropriate tomogram will be described later.

It can be seen that the average and standard deviation are better in a case where the contact is taken into account than in a case where the contact is not taken into account, and a statistically significant difference is obtained between the median of the two at a significance level of 0.001. Therefore, it was suggested that accuracy could be improved by taking into account contact with surrounding organs.

### Second Embodiment

In the following, a second embodiment of the present invention will be described in detail with reference to the accompanying drawings. In the first embodiment, a case where the position and the direction of the tomogram to be captured are determined in advance has been described. In the present embodiment, a case where the position and the direction of the tomogram to be captured are selected will be described. An organ deformation estimation system according to the present embodiment is referred to as an organ deformation estimation system 1a.

The same components as those in the first embodiment described above are denoted by the same reference numerals, and a description of the same components and operations may be omitted.

[Functional Configuration of Organ Deformation Estimation System 1a]

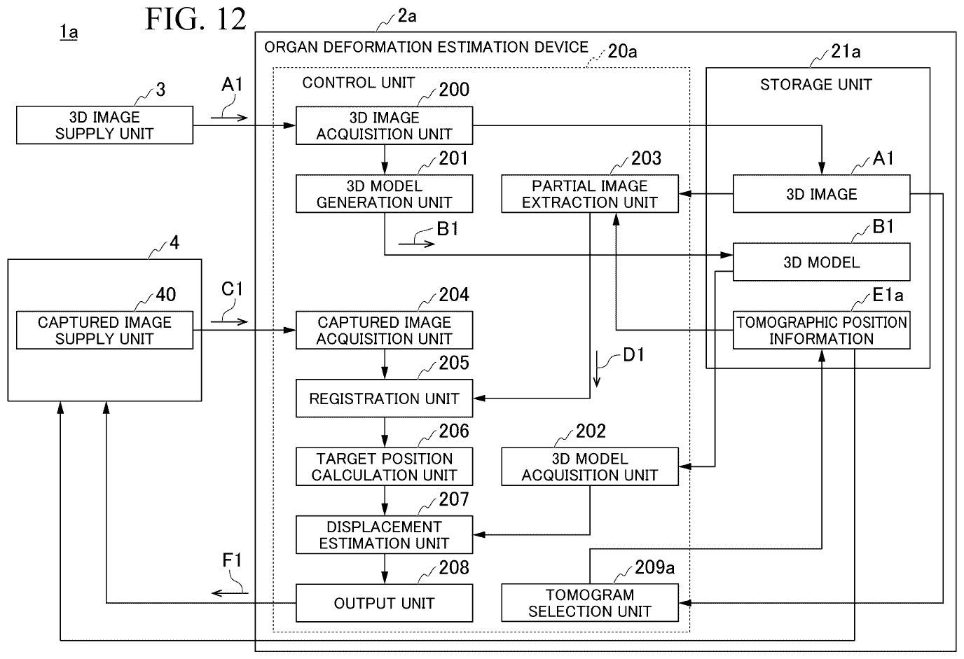

FIG. 12 is a diagram showing an example of a functional configuration of the organ deformation estimation system 1a according to the present embodiment. The organ deformation estimation system 1a includes an organ deformation estimation device 2a, a 3D image supply unit 3, and a captured image supply unit 40.

The organ deformation estimation device 2 includes a control unit 20a and a storage unit 21a. The control unit 20a includes a 3D image acquisition unit 200, a 3D model generation unit 201, a 3D model acquisition unit 202, a partial image extraction unit 203, a captured image acquisition unit 204, a registration unit 205, a target position calculation unit 206, a displacement estimation unit 207, an output unit 208, and a tomogram selection unit 209a. When comparing the control unit 20a (FIG. 12) according to the present embodiment with the control unit 20 (FIG. 3) according to the first embodiment, the tomogram selection unit 209a is different. Here, the functions of the other components (the 3D image acquisition unit 200, the 3D model generation unit 201, the 3D model acquisition unit 202, the partial image extraction unit 203, the captured image acquisition unit 204, the registration unit 205, the target position calculation unit 206, the displacement estimation unit 207, and the output unit 208) are the same as those in the first embodiment.

The tomogram selection unit 209a selects a captured tomogram to be captured as the captured image C1 of the interior of the organ (tomogram). The captured tomogram is a tomogram to be captured as the captured image C1 of the interior (tomogram) of an organ. The captured tomogram is selected by designating a direction of a tomogram and a position in the direction. The tomogram selection unit 209a stores information indicating the selected captured tomogram in the storage unit 21a as a tomographic position information E1a.

### [Tomogram Selection Processing]

The processing of selecting a tomogram by the tomogram selection unit 209a is referred to as tomogram selection processing. The tomogram selection unit 209a executes the tomogram selection processing at a time after the pre-treatment processing and before the calibration.

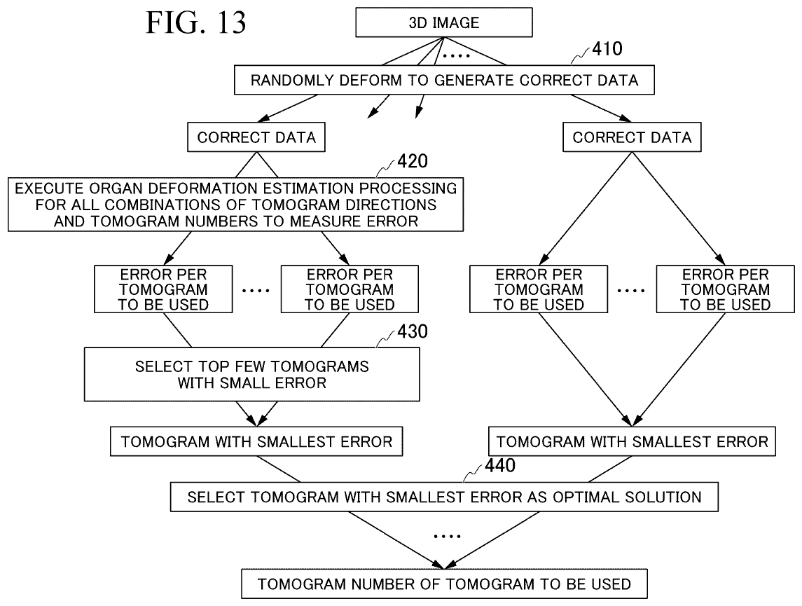

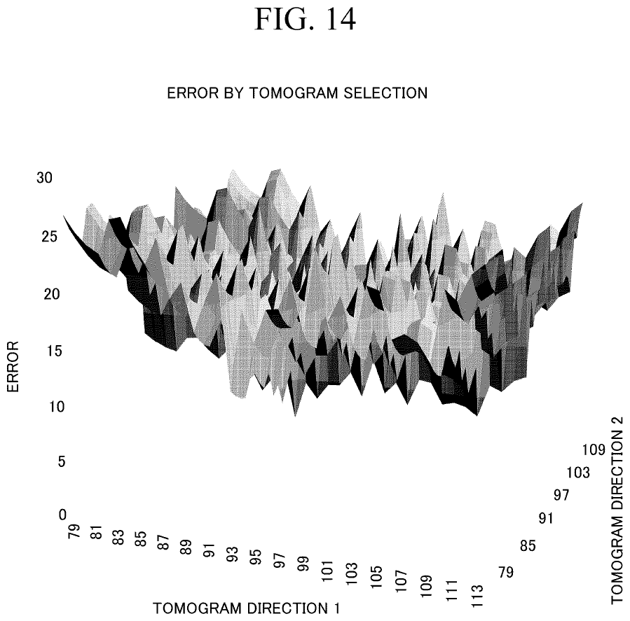

Here, the tomogram selection processing will be described with reference to FIGS. 13 and 14. The tomographic images that can be acquired include a case where the number of candidate captured tomograms is finite and can be fully searched, a case where the number of candidate captured tomograms candidates is finite and cannot be fully searched, and a case where the number of candidate captured tomograms is not finite. The tomogram selection processing is different in each case.

FIG. 13 is a diagram showing an example of a flow of tomogram selection processing according to the present embodiment in a case where the number of candidate captured tomograms is finite and can be fully searched.

Step S410: The tomogram selection unit 209a randomly deforms the 3D image A1 to generate correct data. The correct data is a 3D image generated by randomly deforming each part of the organ captured in the 3D image A1 in units of pixels. A predetermined number of patterns are added by using a random number for the random deformation. The tomogram selection unit 209a generates correct data for each random deformation of a predetermined number of patterns. The predetermined number is, for example, five. In FIG. 13, only two pieces of correct data are shown for simplicity, and other pieces of correct data are omitted.

Step S420: The tomogram selection unit 209a measures the error of the result of the organ deformation estimation processing for all combinations of the tomogram directions and the tomogram numbers. The tomogram number is a number assigned to each tomogram at one or more positions in a certain tomogram direction.

The tomogram selection unit 209a extracts a 2D image from the correct data for a tomogram designated by a certain tomogram direction and a certain tomogram number. The control unit 20a executes the organ deformation estimation processing by using the extracted 2D image instead of the captured image C1. The tomogram selection unit 209a measures an error of the estimation result of deformation of the organ by the organ deformation estimation processing, with respect to the correct data.

For example, in a case where there are two types of tomogram directions and the number of tomograms is 30, all combinations of the tomogram directions and the tomogram numbers are 900. In a case where the number of combinations is enormous, the number of combinations may be reduced by using the other parameter tuning methods instead of executing the organ deformation estimation processing for all the combinations. The other parameter tuning methods are, for example, random search or Bayesian optimization.

Step S430: The tomogram selection unit 209a selects the top few tomograms (for example, the top 5 tomograms) with the smallest measured error.

Each of the processing of step S420 and step S430 described above is executed for each correct data. Step S440: The tomogram selection unit 209a selects the tomogram with the smallest error as the optimal solution (the captured tomogram) with respect to the group of tomograms selected in step S430. The tomogram selection unit 209a, for example, selects the tomogram with the smallest error by weighted averaging by rank. The tomogram selection unit 209a, for example, stores the tomogram number of the captured tomogram in the storage unit 21a as the tomographic position information E1.

FIG. 14 is a diagram showing an example of tomogram selection processing according to the present embodiment in a case where the number of candidate captured tomograms is finite and cannot be fully searched or in a case where the number of candidate captured tomograms is not finite. A case where the number of candidate captured tomograms is finite and cannot be fully searched includes, for example, a case where the number of candidate captured tomograms is enormous. In addition, in a case where the number of candidate captured tomograms is not finite, for example, even a case where the position of a candidate captured tomogram is not included in the 3D image A1 includes a case where the tomogram is virtually defined by using image interpolation or the like and used as a candidate captured tomogram. Therefore, the candidate captured tomograms may include not only tomograms that correspond to positions included in the 3D image A1 captured in advance, but also tomograms that correspond to positions not included in the 3D image A1.

In FIG. 14, the error of the estimation result with respect to the correct data is shown for two tomogram directions of a “tomogram direction 1” and a “tomogram direction 2”. The estimation result is a result obtained by the same processing as in step S420 described above. Among the errors shown in FIG. 14, the 2D image used instead of the captured image C1 to execute the organ deformation estimation processing is, for example, a 2D image generated from the 3D image A1 by using image interpolation or the like. In a case where the number of candidate captured tomograms is finite and cannot be fully searched, or in a case where the number of candidate captured tomograms is not finite, the tomogram selection unit 209a searches for an optimal cross-section based on an optimization method. The optimization method is, for example, a stochastic gradient descent method.

As described above, the organ deformation estimation device 2a according to the present embodiment includes the tomogram selection unit 209a. The tomogram selection unit 209a selects the captured tomogram from among the candidate tomograms for one or more tomogram directions and one or more positions in the tomogram directions, based on an error of an estimation result of deformation of the organ estimated in a case where a 2D image acquired for the candidate tomograms is used instead of the captured image C1 from correct data, which is a 3D image obtained by randomly deforming the 3D image A1, with respect to the correct data. With this configuration, in the organ deformation estimation device 2a according to the present embodiment, it is possible to select a tomogram in which the error of the estimation result of the deformation of the organ is small, and thus the error of the estimation result can be reduced.

The method of selecting an appropriate tomogram is not limited to the tomogram selection processing described in the present embodiment. As a method of selecting an appropriate tomogram, a method may be used, in which a regression model is created in which the tomographic position is used as an explanatory variable and the registration error, which is an evaluation index, or the value and rank related to the Dice coefficient are used as objective variables, to obtain the optimal tomographic position. When the above regression model was actually created, a highly accurate regression model with a coefficient of determination of 0.903 to 0.951 was created. The execution time was 1.57±0.213 ms, and it was confirmed that tomogram selection could be performed in real time during the treatment.

### Second Example

A second example which is an example according to the second embodiment will be described. In the present example, five cases generated by randomly deforming one 3D image by using random numbers were used as correct data to perform a 3D simulation for all tomograms. In the 3D simulation, three organs of the pancreas, stomach, and duodenum were discretized, and a combination in which the estimation error was small was also examined.

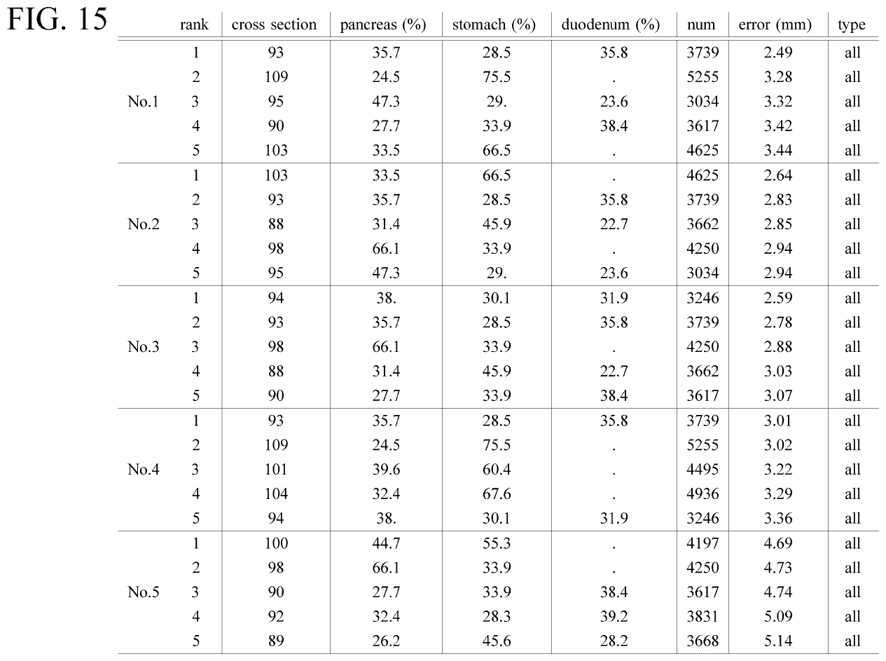

FIG. 15 shows the errors and tomogram numbers for the top five tomograms in ascending order of error for each of the five cases of correct data. The tomogram numbers are 79 to 113, and the median of the tomogram numbers is 96. In the present example, the following four types of organ combinations (called “types”) were used. The first type of combination is a case where the pancreas, stomach, and duodenum were all discretized (called “all”). The two types of combination is a case where the pancreas and stomach are discretized (called “w/o duo”). The third type of combination is a case where the pancreas and duodenum are discretized (called “w/o stom”). The fourth type of combination is a case where only the pancreas is discretized (called “panc”).

In FIG. 15, each column of “pancreas”, “stomach”, and “duodenum” indicates a proportion of the area of each organ on the tomogram. There are many blank spaces in the duodenum area, but this indicates that the duodenum is discretized as the 3D model B1 but does not exist on the tomogram because the duodenum is smaller in area than the pancreas and stomach.

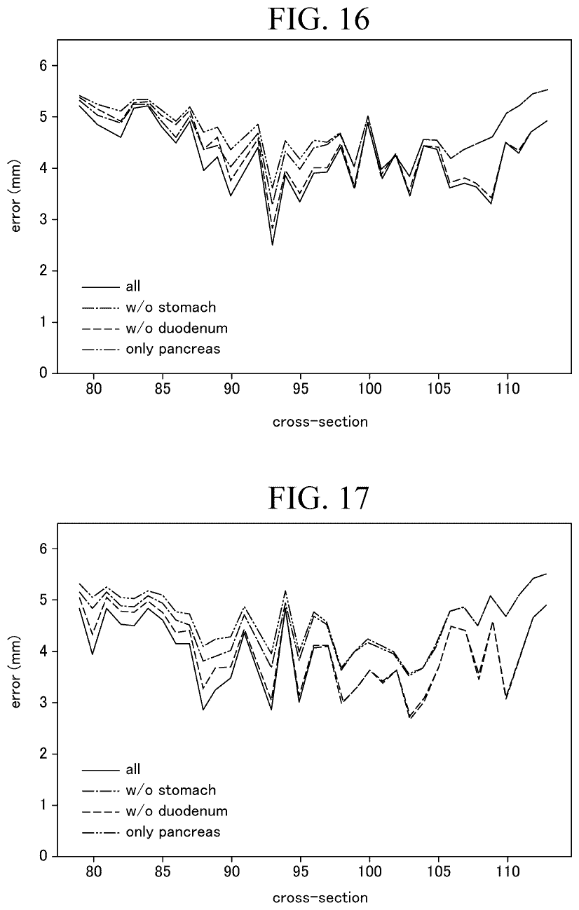

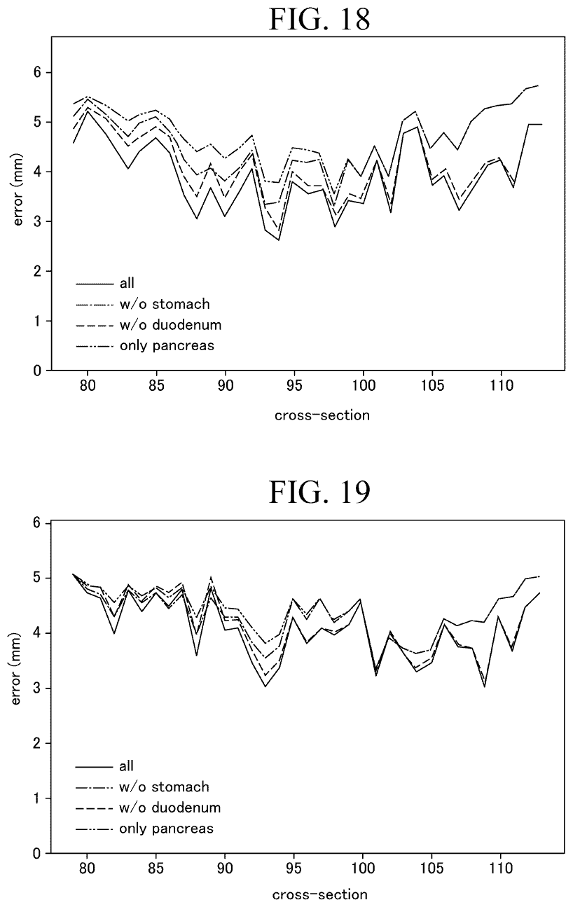

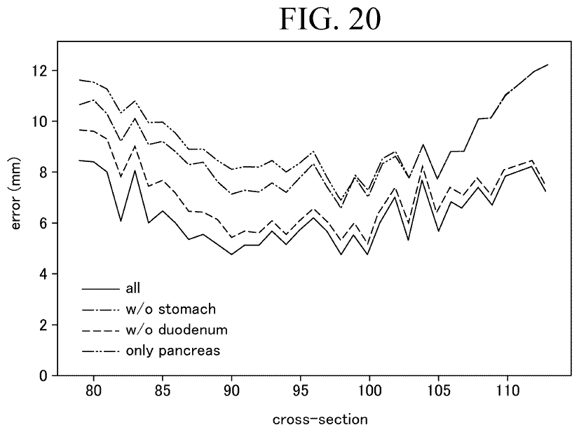

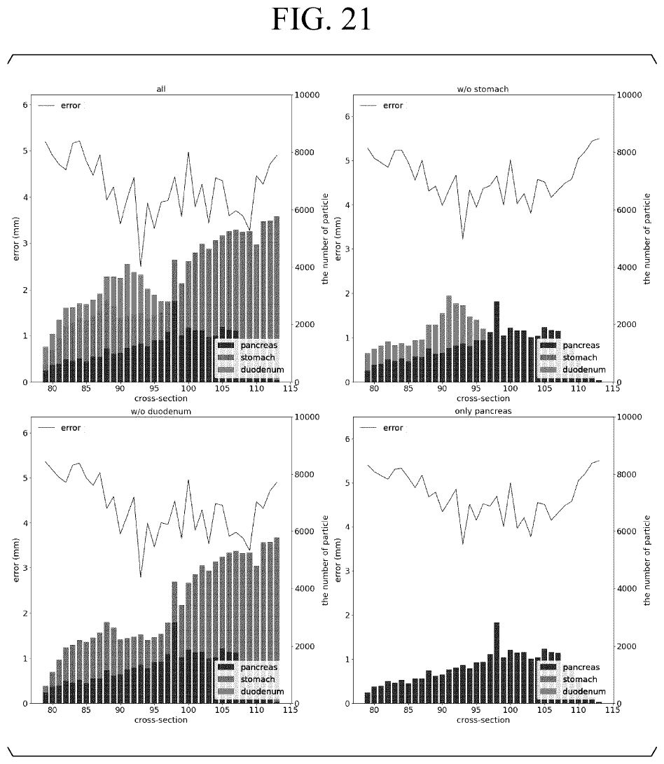

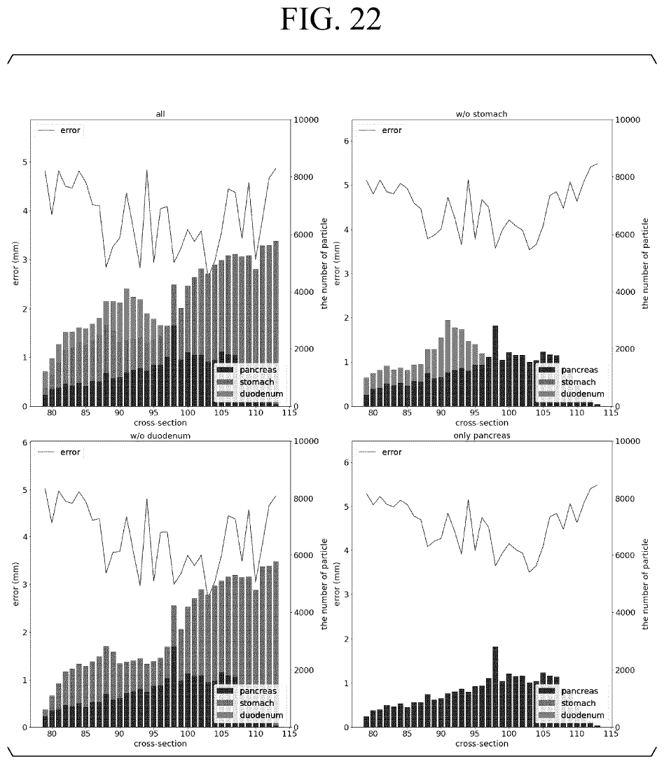

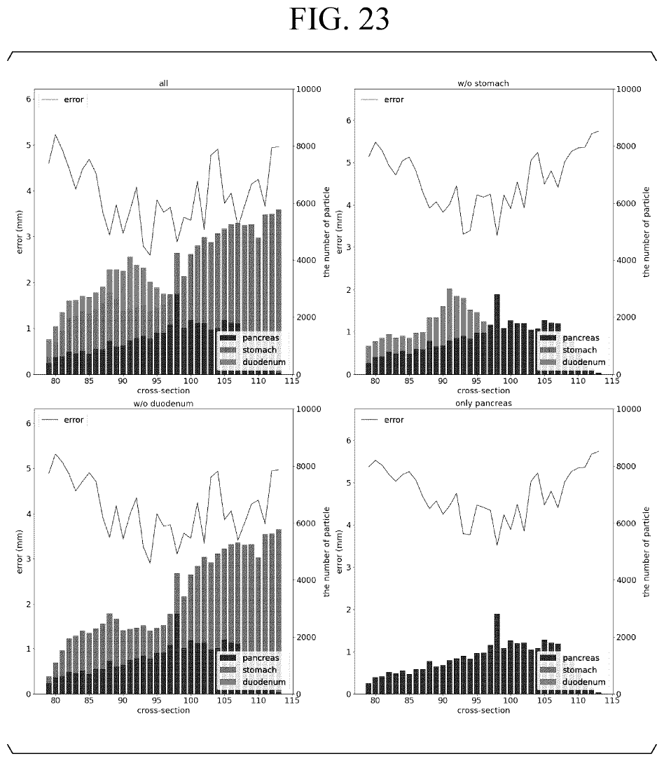

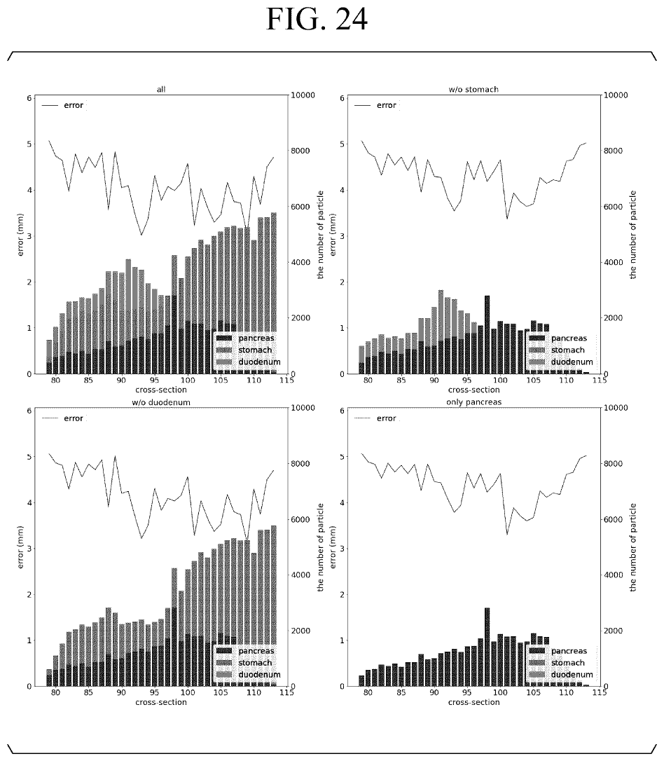

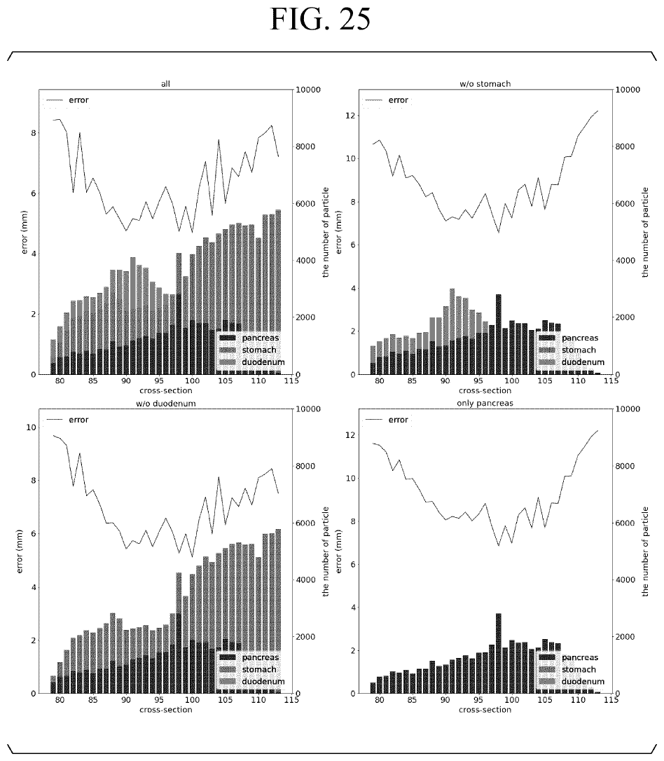

For each example, graphs of the transition of the error in a case where the tomogram number is changed are shown in FIGS. 16 to 20. FIGS. 21 to 25 are graphs showing a relationship between the transition of the error and the number of particles in a case where the tomogram number is changed. From the results of the present example, it was confirmed that the accuracy of estimating the deformation of an organ increases as the number of organs used in the 3D simulation increases.

In the above-described embodiment, an example of a case where the organ deformation estimation devices 2 and 2a are used in radiotherapy has been described, but is not limited thereto. The organ deformation estimation devices 2 and 2a may be used for treatment other than radiotherapy or for treatment support. The treatment other than radiotherapy is, for example, a heavy ion therapy or a surgery as a surgical treatment. In addition, the treatment support is, for example, image-guided surgery support, robot-assisted surgery, and the like. In addition, the organ deformation estimation devices 2 and 2a may be used for various treatments as a treatment device or a treatment system including the organ deformation estimation devices 2 and 2a. In addition, the organ deformation estimation devices 2 and 2a may be used for supporting various treatments as a treatment support device or a treatment support system including the organ deformation estimation devices 2 and 2a. An example of the treatment system is a radiotherapy system including the organ deformation estimation device 2, an imaging device, and a radiation irradiation device.

A part of the organ deformation estimation devices 2 and 2a in the above-described embodiment, for example, the 3D image acquisition unit 200, the 3D model generation unit 201, the 3D model acquisition unit 202, the partial image extraction unit 203, the captured image acquisition unit 204, the registration unit 205, the target position calculation unit 206, the displacement estimation unit 207, the output unit 208, and the tomogram selection unit 209a, may be realized by a computer. In that case, the program to realize this control function may be realized by recording the program to a computer-readable recording medium and having a computer system read and execute the program recorded on this recording medium. The “computer system” here is a computer system incorporated in the organ deformation estimation devices 2 and 2a, and includes an OS and hardware such as peripheral devices. In addition, a term “computer-readable recording medium” refers to a storage device, for example, a portable medium such as a flexible disk, a magneto-optical disk, a ROM, or a CD-ROM, a hard disk built in a computer system, or the like. Further, the term “computer-readable recording medium” may also include a medium that dynamically holds the program for a short time, such as a communication line in a case where the program is transmitted via a network, such as the Internet, or a communication channel, such as a telephone line, and a medium that holds the program for a certain time, such as a volatile memory inside the computer system as the server or the client in that case. In addition, the program may be a program realizing a part of the functions described above, further, the functions described above may be realizable by combining with the program already recorded in the computer system. In addition, a part or all of the organ deformation estimation devices 2 and 2a in the above-described embodiment may be realized as an integrated circuit such as a large scale integration (LSI). Each functional block of the organ deformation estimation devices 2 and 2a may be implemented as a separate processor, or some or all of the functional blocks may be integrated into a processor. Also, the integrated circuit making method is not limited to the LSI, but may be realized by a dedicated circuit or a general-purpose processor. In addition, when the integrated circuit making technology that replaces the LSI appears due to advances in semiconductor technology, an integrated circuit based on the technology may be used.

In the above, although the embodiments of the present invention have been described in detail with reference to the drawings, a specific configuration is not limited to the above, and various design changes and the like can be made without departing from the gist of the present invention.

### REFERENCE SIGNS LIST

2, 2a organ deformation estimation device
200 3D image acquisition unit
202 3D model acquisition unit
203 partial image extraction unit
203 partial image extraction unit
204 captured image acquisition unit
205 registration unit
206 target position calculation unit
207 displacement estimation unit
A1 3D image
B1, B11, B12, B21, B22, B31, B32 3D model
C1, C10, C11 captured image
D1 partial image

1. An organ deformation estimation device, comprising:
a memory; and
a processing unit connected to the memory that:
acquires a 3D model generated based on a 3D image of an organ captured in advance at a time before a treatment is performed and showing a shape of the organ at the time before the treatment is performed;
acquires a captured image which is a 2D image in which an interior of the organ is captured in a course of the treatment;
extracts a partial image which is a 2D image of a part that corresponds to a position of the interior captured in the captured image among parts included in the 3D image;
performs registration between pixels constituting the captured image and pixels constituting the partial image such that pixels indicating the same part of the interior correspond to each other;
calculates a target position of displacement of a part that corresponds to the interior among parts constituting the 3D model based on a result of the registration; and
estimates deformation of the organ in the course of the treatment by deforming the 3D model based on a 3D simulation so as to displace a position of the part that corresponds to the interior among parts constituting the 3D model to the target position.

2. The organ deformation estimation device according to claim 1, wherein
the organ consists of a target organ of the treatment and other organs adjacent to the target organ.

3. The organ deformation estimation device according to claim 1, wherein
processing unit acquires the 3D image.

4. The organ deformation estimation device according to claim 1, wherein
processing unit performs the 3D simulation based on a mesh-free method.

5. The organ deformation estimation device according to claim 1, wherein
the processing unit selects a captured tomogram to be captured as the captured image of the interior of the organ.

6. The organ deformation estimation device according to claim 5, wherein
the processing unit selects the captured tomogram, from among candidate captured tomograms for one or more tomogram directions and one or more positions in the tomogram directions, based on an error of an estimation result of deformation of the organ estimated in a case where a 2D image acquired for the candidate tomograms from correct data, which is a 3D image obtained by randomly deforming the 3D image, is used instead of the captured image with respect to the correct data.

7. A treatment device comprising:
the organ deformation estimation device according to claim 1.

8. A treatment support device comprising:
the organ deformation estimation device according to claim 1.

9. An organ deformation estimation method, comprising:
acquiring a 3D model generated based on a 3D image of an organ captured in advance at a time before a treatment is performed and showing a shape of the organ at the time before the treatment is performed;
acquiring a captured image which is a 2D image in which an interior of the organ is captured in a course of the treatment;
extracting a partial image which is a 2D image of a part that corresponds to a position of the interior captured in the captured image among parts included in the 3D image;
performing registration between pixels constituting the captured image and pixels constituting the partial image such that pixels indicating the same part of the interior correspond to each other;
calculating a target position of displacement of a part that corresponds to the interior among parts constituting the 3D model based on a result of the registration; and
estimating deformation of the organ in the course of the treatment by deforming the 3D model based on a 3D simulation so as to displace a position of the part that corresponds to the interior among parts constituting the 3D model to the target position.

10. A non-transitory computer-readable medium storing instructions which, when executed by a computer, cause the computer to execute:
acquiring a 3D model generated based on a 3D image of an organ captured in advance at a time before a treatment is performed and showing a shape of the organ at the time before the treatment is performed;
acquiring a captured image which is a 2D image in which an interior of the organ is captured in a course of the treatment;
extracting a partial image which is a 2D image of a part that corresponds to a position of the interior in the captured image among parts included in the 3D image;
performing registration between pixels constituting the captured image and pixels constituting the partial image such that pixels indicating the same part of the interior correspond to each other;
calculating a target position of displacement of a part that corresponds to the interior among parts constituting the 3D model based on a result of the registration; and
estimating deformation of the organ in the course of the treatment by deforming the 3D model based on a 3D simulation so as to displace a position of the part that corresponds to the interior among parts constituting the 3D model to the target position.
